(19) United States
(12) Patent Application Publication
(10) Pub. No.: US 2025/0084020 A1
(43) Pub. Date: Mar. 13, 2025

(54) CATALYSTS FOR OXYCHLORINATION OF ETHYLENE TO 1,2-DICHLOROETHANE AND METHODS OF PREPARATION THEREOF

(71) Applicant: BASF Corporation, Florham Park, NJ (US)

(72) Inventors: Jonglack KIM, Ludwigshafen (DE); Joseph John ZAKZESKI, Ludwigshafen (DE); Elena PARVULESCU, Ludwigshafen (DE); Hannes BISCHOF, Ludwigshafen (DE); Rolf TOMPERS, Ludwigshafen (DE); Keith KRAMER, Wichita, KS (US)

(21) Appl. No.: 18/729,719

(22) PCT Filed: Jan. 19, 2023
(86) PCT No.: PCT/US2023/011116

§ 371 Date: Jul. 17, 2024

Related U.S. Application Data
(60) Provisional application No. 63/300,870, filed on Jan. 19, 2022.

(51) Int. Cl. C07C 17/357; B01J 35/61; B01J 37/02; B01J 37/08

(52) U.S. Cl. CPC C07C 17/357; B01J 35/613; B01J 35/615; B01J 37/0213; B01J 37/08; C07C 2521/06; C07C 2521/10; C07C 2523/72

(57) ABSTRACT

Disclosed are catalyst compositions for the oxychlorination of ethylene to dichloroethane (DCE) capable of providing high ethylene conversion, high dichloroethane selectivity and high crude purity. Also disclosed are methods of preparing and using the catalyst compositions. Described herein is a catalyst composition comprising a support and catalytic metal compounds on the support. The catalytic metal compounds can include a copper compound; and a zirconium compound.

### RELATED APPLICATIONS

This application claims priority to U.S. Provisional Patent Application No. 63/300,870, filed Jan. 19, 2022, which is herein incorporated by reference in its entirety.

### FIELD

The disclosure relates generally to catalysts for the oxychlorination of ethylene to 1,2-dichloroethane (DCE) capable of providing high ethylene conversion, high dichloroethane selectivity and high DCE crude purity. The disclosure also relates to methods of preparation of such catalysts.

### BACKGROUND

The most commonly used process for producing 1,2-dichloroethane is the oxychlorination of ethylene in which ethylene is converted with hydrochloric acid (HCl) and oxygen (or an oxygen containing gas) to form 1,2-dichloroethane and water. For this reaction, fixed bed and fluidized bed processes have been developed and are currently in use. Various processes based on this reaction operate using air or an oxygen-enriched gas such as more or less pure oxygen: the later can contain little amount of impurities, for example, Ar, N₂, CO, CO₂ and so on.

In processes using fixed bed reactors, temperature control of the fixed bed is difficult because of localized hot spots in the tubes. In processes using fluidized bed oxychlorination reactors, the use of certain catalysts within the reactors may result in catalyst sticking, especially at relatively low temperature or high HCl partial pressure, which results in the agglomeration of catalyst particles losing fluidizability of catalyst and/or the plugging of the cyclones.

### BRIEF DESCRIPTION OF THE DRAWINGS

The drawings, described below, are for illustrative purposes. The drawings are not intended to limit the scope of the disclosure in any way.

FIG. 1 is a chart showing the DCE selectivity of catalysts according to the invention (Examples 1-5) with respect to comparative catalysts (Comparative Examples 1-4) when measured at a temperature of about 220-240° C.

FIG. 2 is a chart showing the selectivity to chlorine byproducts of catalysts according to the invention (Examples 1-5) with respect to comparative catalysts (Comparative Examples 1-4) when measured at a temperature of about 220-240° C.

FIG. 3 is a chart showing the DCE purity of catalysts according to the invention (Examples 1-5) with respect to comparative catalysts (Comparative Examples 1-4) when measured at a temperature of about 220-240° C.

FIG. 4 is a chart showing the ethylene conversion of catalysts according to the invention (Examples 1-5) with respect to comparative catalysts (Comparative Examples 1-4) when measured at a temperature of about 220-240° C.

FIG. 5 is a chart showing the hydrochloric acid conversion of catalysts according to the invention (Examples 1-5) with respect to comparative catalysts (Comparative Examples 1-4) when measured at a temperature of about 220-240° C.

### SUMMARY

According to embodiments, described herein is a catalyst composition comprising a support and catalytic metal compounds on the support. The catalytic metal compounds can include a copper compound; and a zirconium compound, wherein the support comprises a BET surface of about 50 m²/g to about 250 m²/g.

According to further embodiments described herein is a method of forming a catalyst composition, comprising: dissolving (a) a copper compound, and (b) a zirconium compound in a solvent with agitation and at a temperature of about 20° C. to about 80° C. to form a solution; dosing the solution onto a support comprising a BET surface of about 50 m²/g to about 250 m²/g, wherein the support is rotated at about 50 rotations per minute (rpm) to about 150 rpm; and drying the coated support to remove free water for about 1 hour to about 20 hours at a temperature of about 75° C. to about 180° C.

### DETAILED DESCRIPTION

It is an object of the present invention to provide a catalyst for oxychlorination of ethylene to DCE that provides high activity (allowing low reaction temperature), excellent DCE selectivity and especially high DCE crude purity (a low selectivity of undesired chlorinated by-products). When such a catalyst is used in powder form in a fluidized bed process, it is a further object to allow operation of the catalyst under industrial conditions with minimum risk of sticking.

Described herein are catalyst compositions and methods of manufacturing thereof for use in the oxychlorination of ethylene to 1,2-dichloroethane (DCE). According to embodiments, the catalyst compositions contain a copper compound, an alkaline earth metal compound comprising magnesium and a transition metal compound comprising zirconium. It has been found surprisingly that it is possible to obtain catalysts for the oxychlorination of ethylene to DCE that are capable of improving DCE product purity because of reduced selectivity toward chlorinated by-products with high ethylene conversion.

When air is used as oxygen-containing gas, industrial processes are often operated in a single-pass mode without a recycling stream to send some part of the reactor effluent to the reactor inlet. In that case, when air is used, ethylene and hydrogen chloride are typically fed to the reactor in roughly stoichiometric ratio, allowing close to full conversion of both ethylene and hydrogen chloride. In practice, typically a slight excess of ethylene is applied in order to minimize the level of non-converted hydrogen chloride in the reactor effluent. When more ore or less pure oxygen is used as oxygen-containing gas, in general a very different ratio of ethylene and hydrogen chloride is fed to the reactor. Typically, a huge excess of ethylene is applied, allowing nearly full conversion of hydrogen chloride but only partial conversion of ethylene. After separation of EDC, hydrogen chloride, some water and by-products, the remaining gas stream, after removal of a purge stream is recycled to the reactor. In this recycle process, per pass conversion of ethylene is typically kept quite low (e.g., less than about 90% to less than about 96%).

The catalysts used in oxychlorination processes can contain copper chloride as an active ingredient. In order to improve the activity, selectivity and/or the operability, further promoters may be introduced into the catalyst formulation. Among the most commonly used are magnesium chloride, potassium chloride, cesium chloride and/or rare earth chlorides. The active copper species as well as the promoters can be deposited on a high surface area support such has kieselguhr, clay, fuller's earth, silica or alumina. In general, the copper and the promoters are impregnated onto the support by means of a solution containing all the metals in form of their chlorides. In some cases, a co-precipitation of the ingredients with the support is carried out.

In fixed bed reactors, catalyst is packed in vertical alloy tubes held in a tube sheet at top and bottom. Fixed bed catalysts typically are shaped bodies such as e.g. small pellets, granules, cylinders or hollow-cylinders (rings). Uniform packing of theses catalysts within the tubes is of importance to ensure uniform pressure drop, flow, and residence time through each tube. Suitable catalyst shapes and sizes depend on the particular reactor used. However, temperature control is still difficult in a fixed bed because of the development of localized hot spots in the tubes.

In order to minimize the formation of hot spots, inert diluent is mixed with catalyst pellet in proportions that vary along the length of the tubes to achieve low catalyst activity at the inlet, but highest at the outlet. As an alternative, the tubes are filled with catalysts having a progressively higher loading of copper compound so as to form an activity gradient along the length of the tubes.

Multiple reactors in series are also used in fixed bed oxychlorination, primarily to control heat release by staging the air or oxygen feed. Each successive reactor may also contain catalyst with a progressively higher loading of cupric chloride. In general, fixed bed oxychlorination operates at higher temperatures (230-300° C.) and gauge pressures (150-1400 kPa). Fixed bed reactors have a finite catalyst life due to fouling or coking of the catalyst bed, which requires periodic, complete catalyst replacement.

Fluidized bed oxychlorination reactors typically are vertical cylindrical vessels equipped with a support grid and feed sparger system designed to provide good fluidization and feed distribution. Fluidized bed catalysts are fine powders having a particle size ranging from about 20 microns to about 200 microns in diameter. Catalyst carryover during the operation is recovered by internal or external cyclones, and reaction heat is removed by internal cooling coils.

Compared to a fixed bed process, a fluidized bed may offer a more homogeneous temperature distribution throughout the reactor because of enhanced transport characteristics and can operate at lower pressures and temperatures. A typical operating temperature and gauge pressure of fluidized bed reactors are of 220-245° C. and of 150-500 kPa, respectively. Commercial fluidized bed reactors can be operated with an HCl conversion of 99.5 to 99.8%. The 1,2-dichloroethane selectivity typically lies between 96 and 97.5%.

In contrast to fixed bed reactors, fluidized bed reactors with some catalysts may suffer from catalyst sticking, especially at relatively low temperature or high HCl partial pressure, which results in the agglomeration of catalyst particles losing fluidizability of catalyst and/or the plugging of the cyclones. Since such a sticking of catalyst causes significant economic damage to a production plant, it has to be avoided by all means. Stickiness of catalyst can be caused either by inappropriate operational conditions or by the properties of the catalyst itself. It is known that addition of alkali metal and/or alkali earth metal, e.g. potassium and magnesium, to catalyst formulation can improve resistance of catalyst to stickiness.

The by-products formed in the oxychlorination process are carbon oxides (CO+CO₂) and chlorinated hydrocarbons. Among these chlorinated by-products 1,1,2-trichloroethane, choral, ethylchloride, chloroform and carbon tetrachloride are the most common. In general, selectivites to carbon oxides and chlorinated by-products increases as operation temperature rises. As all by-products lead to a loss in ethylene efficiency, selectivities to theses by-products in oxychlorination of ethylene have to be minimized. CO and CO₂ are easily separated from the DCE product, and thus, do not negatively impact product quality, however, the heavy chlorinated organic by-products are not easily separated. These heavy chlorinated organic by-products can be harmful and their presence in the DCE product reduces the DCE product quality and value.

It is to be understood that the invention is not limited to the details of construction or process steps set forth in the following description. The invention is capable of other embodiments and of being practiced or being carried out in a variety of ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a catalyst material” includes a single catalyst material as well as a mixture of two or more different catalyst materials.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Unless otherwise indicated, all parts and percentages are by weight. Weight percent (wt. %), if not otherwise indicated, is based on an entire composition.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that are within the scope of the appended claims and their equivalents.

### Catalyst Compositions

Catalyst compositions as described herein improve DCE selectivity and product purity while achieving high ethylene and HCl conversion. In particular, the lower quantities of undesired heavy chlorinated by-products increase product quality and value while reducing purification costs and protecting the environment. In some embodiments, catalyst compositions according to embodiments herein have an ethylene dichloride selectivity of about 95 mol % to about 99.9 mol % when contacted with hydrochloric acid, ethylene and oxygen at a temperature of about 200° C. to about 250° C.

According to one or more embodiments, the catalyst compositions described herein employ support materials. For fluid bed catalysis, the metals can be deposited on a support, for example, a high surface area support. One reason for using a high surface area support is to reduce stickiness of the catalyst as the metal can be dispersed over a large area.

Examples of high surface area support materials include, but are not limited to, silica, magnesia, kieselguhr, clay, fuller's earth, alumina, zeolites or combinations thereof. According one or more embodiments, the support can be a high surface area powder (e.g., gamma, delta or theta alumina powder), for example, suitable for a fluidizable support, comprised of particles, granules and/or spheres (e.g., alumina microspheres or nanospheres in amorphous or colloidal form), which may be referred to herein as “supports.” In embodiments, alumina supports can be activated or transition aluminas generated by calcination of a hydrated or hydroxylated precursor alumina. These activated aluminas can be identified by their ordered structures observable by their X-ray diffraction patterns, which indicate a mixed phase material containing minimal to no low surface area or crystalline phase alpha alumina. In some embodiments, the support may be in suitable form for a fixed bed reactor and may be in the form of tablets and/or extrudates. For example, the tablets or extrudates can be formed of compressed or extruded particles, granules and/or spheres as described above. The tablets and/or extrudates can have a cross-section dimension of about 1 mm to about 100 mm, or any suitable value or sub-range within this range.

Alpha alumina is identified by a defined crystalline phase by x-ray diffraction. The higher surface area activated aluminas are often defined as a transition alumina, but the phase transitions can be a continuum of varying percentages of multiple mixed phases such as, but not limited to, gamma, delta and theta phases based on the chosen calcination temperature to achieve the desired support surface area. According to embodiments, a gamma alumina support can be used. In embodiments, the gamma alumina support is a powder having a gamma phase of at least about 95%, or at least about 98%, or at least about 99%, or at least about 99.995% and a surface area of about 50 m²/g to about 250 m²/g, or about 70 m²/g to about 225 m²/g, or about 100 m²/g to about 215 m²/g, or about 150 m²/g to about 205 m²/g. According to embodiments, the support (e.g., gamma alumina powder) has a surface area of at least about 80 m²/g in order to properly disperse the metal load and reduce the tendency for stickiness. The catalysts will be described hereinafter in terms of a fluidizable alumina support (e.g., a gamma alumina powder). This is meant to be illustrative and not limiting.

According to embodiments, the alumina support material has a compacted bulk density of about 0.5 g/cm³ to about 4.0 g/cm³, or about 0.75 g/cm³ to about 3.0 cm³, or about 1.0 cm³ to about 2.0 cm³, or about 0.7 cm³ to about 1.3 cm³, or about 3.65 cm³. The fluidizable alumina support material can have a pore volume of about 0.1 cm³/g to about 2 cm³/g, or about 0.2 cm³/g to about 1 cm³/g, or about 0.3 cm³/g to about 0.75 cm³/g. According to embodiments, the fluidizable alumina support material can have a particle size distribution such that (a) about 90 to about 100 percent by volume of the particles are less than about 150 μm, or less than about 140 μm, or less than about 125 μm, or less than about 110 μm in diameter; (b) about 50 to about 60 percent by volume of the particles are less than about 75 μm, or less than about 70 μm, or less than about 65 μm, or less than about 60 μm; and (c) about 20 to about 35 percent by volume of the particles are less than about 45 μm, or less than about 40 μm, or less than about 35 μm, less than about 30 μm, as measured by a Malvern Instruments, Ltd. Fraunhofer laser diffraction (i.e., light scattering) analyzer where, at room temperature (about 20° C. to about 25° C.) and pressure (about 1 atm), 5 g samples of the material are dispersed in 100 mL of deionized water with a magnetic stirrer for 1 min to form a suspension and 4 mL of the suspension is diluted with 130 mL of deionized water and analyzed at 2500 rotations/min with the analyzer (at room temperature and pressure). Such alumina support materials are readily fluidizable, relatively stable, mechanically strong and resistant to attrition. Aluminas useful as supports are, for example, the Sasol products Puralox® SCCa-57/170 and Catalox® SCCa-25/200 high purity activated aluminas.

Additionally, the alumina support can be stabilized by any means in the art to prevent undesirable changes in the activated alumina phase once the finished catalyst is in service. Examples of such stabilizing components include, but are not limited to, lanthanum (La), praseodymium (Pr), cerium (Ce), silicon (Si), etc. as trace components dispersed throughout the alumina support prior to impregnation of the active catalyst formulation. For example, the alumina support can include about 0.005 wt. % to about 0.50 wt. %, or any individual value or sub-range within this range, of one or more of the stabilizing components.

It is recognized that some alumina support materials may contain, in addition to aluminum oxide (Al₂O₃), small amounts of impurities of other metals such as metal oxides, for example, less than about 0.02 wt. % of sodium oxide, less than about 0.05 wt. % of iron oxide (Fe₂O₃), less than about 0.3 wt. % of titanium dioxide, less than about 0.2 wt. % of silicon dioxide, etc. Ti compounds may remain in the alumina unintendedly from the alumina production process without exerting major effect on physicochemical properties of the latter. In some embodiments, the support is free or substantially free of titanium, for example, the support may contain less than 1500 ppm (by weight), less than 1000 ppm, less than 500 ppm or 0 ppm titanium, or any individual value or sub-range within these ranges.

In some embodiments, the catalyst composition is free or substantially free of silica. For example, in some embodiments, the support does not contain silica and/or the composition as a whole does not contain silica or contains less than 500 ppm, less than 400 ppm, less than 300 ppm, less than 200 ppm, less than 100 ppm (by weight) silica, or any individual value or sub-range within these ranges. Without being bound by any particular theory, it is believed that silica may reduce the activity of the catalyst in the oxychlorination of ethylene to dichloroethane.

In some embodiments, the support is not activated and/or the support does not include activated alumina. For example, prior to impregnation and/or adsorption of the catalytic metals, the support is not heated at a high temperature, for example, a temperature exceeding about 100° C., about 200° C., about 300° C., about 400° C., about 500° C., about 600° C., about 700° C., about 1000° C., or about 600° C. to about 1200° C., or any individual value or sub-range within these ranges. Without being bound by any particular theory, it is believed that pre-heating the support at a high temperature can reduce the BET surface area of the support by about 25% to about 75%, for example, by sintering and phase transition. Such reduction in surface area of the support can impact the catalyst's performance in the conversion of ethylene to dichloroethane by an oxychlorination process.

Without being bound by any particular theory, it is believed that only particular ranges of loadings of Cu and at least one transition metal comprising Zr, will result in all of the high performance characteristics described above. Outside of the particular loadings of the active metals, high performance in all respects may not be achieved. For example, increasing the Cu content increases the catalyst activity, but makes the catalyst prone to sticking. Increasing the Mg content makes the catalyst less prone to sticking; however, the additional Mg decreases the catalyst activity and the DCE crude purity. Increasing the Zr content makes the catalyst less prone to sticking, increases DCE product purity, but slightly reduces catalyst activity. Metal loadings outside the disclosed range result in poor catalyst performance in one or more characteristics including catalyst activity (i.e. ethylene and HCl conversion), catalyst selectivity, DCE product purity, and/or stickiness. According to embodiments, catalysts as described herein provide a dichloroethane crude purity of about 99.20 wt. % to about 99.90 wt. %, or any individual value or sub-range within this range, when employed in a process for the oxychlorination of ethylene in an air based fluidized bed reactor without recycle of the effluent.

The copper compound is used in the form of a water soluble salt and can be used in the form of copper chloride. However, other copper salts that can convert to the chloride during the oxychlorination process can also be used, such as the nitrate salt, carbonate salt, the sulfate salt, or other halide salts like the bromide salt. The copper salt is deposited on the alumina support using the same techniques as described above. The amount of copper metal deposited is based on the activity desired and the specific fluidization characteristics of the support for fluid bed catalyst applications.

According to embodiments herein, catalysts as described can include about 1 wt. % to about 10 wt. %, or any individual value or sub-range within this range, of copper metal based on the total weight of the catalyst composition. In embodiments, the copper salt is a copper chloride (e.g., copper II chloride). The minimum amount of copper metal can be about 2.0 wt. %, or about 3.0 wt. %, or any individual value or sub-range within this range, based on the total weight of the catalyst. The maximum amount of copper metal can be about 6.0 wt. %, or about 8.0 wt. %, or about 10 wt. %, or any individual value or sub-range within this range, based on the total weight of the catalyst.

The at least one alkaline earth metal compound comprises magnesium and can include additional alkaline earth metals such as calcium, strontium, barium, or a mixture thereof. In one or more embodiments, the at least one alkaline earth metal compound comprises magnesium and either no or only trace amounts (e.g., less than 1 ppm) of other alkaline earth metals/compounds. The at least one alkaline earth metal is used in the form of a water soluble salt, and can be used in the form of an alkaline earth metal chloride. However, other alkaline earth metal salts that would convert to the chloride salt during the oxychlorination process can also be used, such as the nitrate salt, the carbonate salt, the sulfate salt or other halide salts like the bromide salts. According to embodiments, the at least one alkaline earth metal compound comprises a magnesium salt such as magnesium chloride, magnesium nitrate, magnesium bromate, magnesium carbonate or magnesium sulfate. In embodiments, the at least one alkaline earth metal compound comprises magnesium chloride. The at least one alkaline earth metal can include the magnesium as metal in the range of about 0.5 wt. % to about 5.0 wt. %, or about 0.75 wt. % to about 4.0 wt. %, or about 1.0 wt. %, to about 3.0 wt. % (as the metal), or any individual value or sub-range within these ranges, based on the total weight of the catalyst composition. According to embodiments, the catalyst can include magnesium metal in an amount of less than about 5.0 wt. %, or less than about 4.0 wt. %, or less than about 3.0 wt. %, or any individual value or sub-range within this range, based on the total weight of the catalyst composition. If an additional alkaline earth metal is included, the catalyst can contain about 0.005 wt. % to about 10.0 wt. %, or any individual value or sub-range within this range, of the additional alkaline earth metal.

The at least one transition metal compound comprises zirconium and can include additional transition metals such as Sc, TI, V, Y, Nb, La, Hf, Ta, Ac, Rf, Ha, Mn or a mixture thereof. In one or more embodiments, the at least one transition metal compound comprises zirconium and either no or only trace amounts (e.g., less than 0.05 wt. %) of other transition metals/compounds. In one or more embodiments, the catalyst composition contains zirconium and does not contain manganese (Mn) or rhenium (Re). The at least one transition metal compound can be used in the form of a water soluble salt such as a transition metal chloride (e.g., an oxychloride). However, other salts that would convert to the chloride salt during the oxychlorination process can also be used, such as the nitrate salt, the carbonate salt, the sulfate salt or other halide salts like the bromide salts. According to embodiments, the at least one transition metal compound comprises a zirconium salt such as zirconium chloride, zirconium oxychloride, zirconium chloride, zirconium carbonate or zirconium hydroxide. These zirconium salts can contain about 0.05 wt. % to about 1.0 wt. %, or any individual value or sub-range within this range, of other transition metals as impurity, for example, Hf. The at least one transition metal can include the zirconium as metal in the range about 0.1 wt. % to about 3.0 wt. %, or about 0.5 wt. % to about 2.0 wt. %, or about 0.75 wt. % to about 1.0 wt. %, or any individual value or sub-range within these ranges, based on the total weight of the catalyst composition. According to embodiments, the catalyst can include zirconium metal in an amount of less than about 3.0 wt. %, or less than about 2.0 wt. %, or less than about 1.0 wt. %, or any individual value or sub-range within these ranges, based on the total weight of the catalyst composition. If an additional transition metal is included, the catalyst can contain about 0.005 wt. % to about 10.0 wt. %, or any individual value or sub-range within this range, of the additional transition metal.

The final catalyst composition containing the copper compound, the at least one alkaline earth metal compound comprising magnesium and the at least one transition metal compound comprising zirconium is readily fluidizable. Other metals can be present in the catalyst compositions in relatively small or trace amounts. For example, rare earth metals and/or transition metals other than zirconium. Typically, these metals, if present, may be present in amounts of less than about 0.05 wt. % based on the total weight of the catalyst composition.

### Preparation of Catalyst Compositions

One method of addition of the metals (e.g., Cu, Mg and Zr) onto the alumina support is accomplished by impregnating the support with an aqueous solution of a water soluble salt of the metals and then drying the wetted support. The at least one alkaline earth metal comprising magnesium and at least one transition metal comprising zirconium (and any additional metals) can be, but do not have to be, calcined on the support prior to deposition of the copper compound to produce a fluidizable catalyst. According to embodiments, the desired quantity of metal salt is dissolved in water under vigorous stirring at a temperature of 60° C. Once dissolved, the metal salt solution is slowly introduced to the alumina carrier, for example, in a tumbler rotating at 90 rotations per minute. After the impregnation, the wet catalyst can be dried in a drying oven heated from about 100° C. to about 180° C., or any individual value or sub-range within this range, for about 2 hours to about 30 hours, or about 10 hours to about 28 hours, or about 20 hours to about 25 hours, or any individual value or sub-range within these ranges. The dried catalyst can be sieved hot using a 500 μm sieve.

The specific characteristics such as surface area and pore volume can be modified via the deposition of the metal salts. Catalyst compositions as described herein can have a final surface area in the range of about 20 to about 220 m²/g, or about 60 to about 180 m²/g, or about 70 to about 170 m²/g, or about 80 m²/g to about 150 m²/g, or about 150 m²/g to about 215 m²/g, or any individual value or sub-range within these ranges.

According to embodiments, the catalyst compositions can be prepared by wetting the alumina support material, as above described, with an aqueous solution of salts of the desired metals. The wetted alumina is then dried slowly at a temperature of about 75° C. to about 180° C., or about 90° C. to about 200° C., or about 100° C. to about 180° C., or any individual value or sub-range within these ranges, to remove water. An amount of the metal salt is chosen so that the final catalyst contains from about 1.0% to about 10% by weight of copper, about 0.5 wt. % to about 5.0 wt. % by weight of magnesium and about 0.1 wt. % to about 3.0 wt. % by weight of zirconium, or any individual value or sub-range within these ranges. All metals are calculated based on the total weight of the catalyst composition which includes the catalyst support, metals, and ligands or counter anions associated with any given metal additive. The metal salt used in the aqueous solution can be in the form of any water soluble salt such as previously described, like the chloride or oxychloride salt or any salts known to those of ordinary skill in the art.

The salts used are generally copper chloride (CuCl₂*2H₂O), magnesium chloride (MgCl₂*6H₂O), and zirconium oxychloride (ZrOCl₂*8H₂O). These salts are weighed and then dissolved in deionized water at 60° C. while stirring (e.g., using an impeller at about 100 rpm). The carrier used can be gamma alumina powder with a surface area of about 50 m²/g to about 250 m²/g, or about 70 m²/g to about 225 m²/g, or about 100 m²/g to about 215 m²/g, or about 150 m²/g to about 205 m²/g, a pore volume in the range of 0.3 to about 0.6 cm³/g, and a particle size distribution of about 10 μm to about 115 μm, or about 20 μm to about 110 μm, or about 25 μm to about 100 μm, or about 30 μm to about 80 μm, or any individual value or sub-range within these ranges. The carrier is transferred to a vessel with baffles or a tumbler, which is rotated at about 90 rotations per minute. The salt solution is dosed dropwise onto the carrier within 30 minutes.

After impregnation, the wet catalyst can be dried in an oven for about 1 h to about 10 h, at a temperature of about 100° C. to about 200° C., for example, the wet catalyst can be dried in an oven for about 4 h at 100° C., for 16 h at 110° C., for 2 h at 130° C., for 2 h at 150° C., and for 4 h at 180° C., or any individual value or sub-range within these ranges. The dried catalyst can be sieved hot at a temperature of about 100° C. to about 200° C., for example, a temperature of 130° C. using a 500 μm sieve, or a 475 μm sieve, or a 450 μm sieve, or a 400 μm sieve, or a 350 μm sieve, or a 300 μm sieve, or any individual value or sub-range within these ranges.

### Methods of Using Catalyst Compositions

Also described herein is a method of using catalyst compositions as described herein for the oxychlorination of ethylene to form DCE. The process includes contacting ethylene, oxygen or an oxygen containing gas and hydrogen chloride (HCl) with a catalyst composition as described herein in a reaction zone and recovering the effluent of the reaction zone. The catalyst employed comprises copper, at least one alkaline earth metal comprising magnesium and at least one transition metal comprising zirconium. The metals are deposited on a high surface area support for fluid bed applications.

When the catalyst compositions are initially charged or recharged to an oxychlorination reactor prior to DCE production, the catalyst bed is generally heated and fluidized initially, prior to dose of ethylene, with air, oxygen enriched air, or nitrogen enriched air when operating as a once-through process. For a process where the vent gases are recycled back to the reactor, one can employ either nitrogen, a mixture of oxygen and nitrogen, or a gas mixture containing nitrogen, oxygen, and carbon dioxide.

Catalyst compositions as described herein can also be operated at or above the optimal operating temperature of comparative compositions. However, to take advantage of the ability to produce higher crude purity DCE with sufficient ethylene efficiency, one should operate the catalyst compositions at lower operating temperatures versus comparative compositions. The catalyst compositions as described herein can operate at temperatures of about 0° C. to about 15° C., or about 2° C. to about 10° C., or about 3° C. to about 7° C. lower than comparative compositions. Surprisingly, higher DCE crude product purity is achieved while operating at lower temperatures without the loss of HCl and ethylene conversion or susceptibility to poor fluidization resulting from catalyst stickiness.

The catalyst compositions described herein are highly efficient catalysts for the oxychlorination of ethylene to DCE. The reaction process temperatures can vary from about 180° C. to about 260° C., or from about 210° C. to 250° C. Reaction pressures can vary from about atmospheric to as high as about 200 psig. Contact times in fluid bed and fixed bed catalysis can vary from about 10 seconds to about 50 seconds (contact time is defined as the ratio of reactor volume taken up by the catalyst to the volumetric flow rate of the feed gases at the reactor control temperature and top pressure), and can be from about 20 seconds to about 35 seconds. The ratio of the ethylene, HCl, and oxygen reactants, based on the moles of HCl fed to the reactor, can range from about 1.0 moles to about 2.0 moles of ethylene and about 0.5 mole to about 0.9 mole of oxygen per 2.0 moles of HCl. Typical oxychlorination processes attempt to operate within the stoichiometric ratio of about 1 mole to about 2 moles of HCl to 1 mole of ethylene.

### EXAMPLES

The Examples set forth below illustrate unique and unexpected properties of the catalyst compositions and methods described herein are not intended to be limiting of the invention. The Examples particularly point out the criticality of embodiments using a combination of copper chloride, at least one alkaline earth metal comprising magnesium and at least one transition metal comprising zirconium. In all of the Examples, the fluid bed oxychlorination reaction is conducted using a laboratory scale fluid bed reactor. The reactor volume, the amount of catalyst charged to the reactor, the fluid density, the reactant flow rates, the temperature and the pressure all affect the contact time between reactants and catalyst. Reactor height to diameter ratio can also effect reaction conversions, selectivities, and efficiencies. Therefore, in order to insure that measured differences in catalyst performance results are due strictly to inherent differences in catalyst characteristics rather than to differences in reactor geometry or reactor conditions, all catalyst performance evaluations are conducted in virtually identical laboratory scale reactors using the same reaction contact time, the same set of feed conditions, and the same reactor control methods. The reactor is equipped with means for delivering gaseous ethylene, oxygen, nitrogen, and HCl through the reactor zone, means for controlling the quantities of reactants and reaction conditions, and means for measuring and ascertaining the composition of the effluent gases to determine the percent HCl conversion, percent yield of DCE, and percent ethylene efficiency and DCE product purity.

### Description of Test Reactor

For the test of catalyst performance, a tubular glass reactor with an internal diameter of 2 cm was used. The reactor was operated at 4 bar and was filled with an amount of catalyst leading to a fluidized bed height of about 115 cm. The feed gas was composed of 45.5 NL/h of N₂, 14.95 NL/h of ethylene, 28.40 NL/h of HCl and 10.15 NL/h of O₂: molar ratio of HCl/ethylene, O₂/HCl and O₂/ethylene are 1.9, 0.35 and 0.68, respectively. The reaction temperature was measured with a centered thermocouple in the fluidized bed and regulated on behalf of external electric heating. The reaction temperature range can be widely varied and typically lies between 215 and 240° C. Hydrochloric acid in the feed and in the product gas was measured via titration while N₂, ethylene, O₂, CO and chlorinated hydrocarbons were analyzed by gas chromatography (GC). Based on the analytics and the feed gas amounts, the HCl conversion, the ethylene conversion, the DCE selectivity and the selectivity of the different oxidized and chlorinated by-products were calculated. The chemical performance was evaluated at temperatures above 215° C. where the HCl conversion was generally higher than 97%. The sticking resistance was evaluated by gradually lowering the temperature to either 195° C. or the point where pressure drop across the fluidized bed reaches 100 mbar. Under typical fluidization or non-sticky condition the catalyst was moving freely and smoothly in the reactor with a fairly constant effluent gas exit rate where gaseous pockets or bubbles observed within the bed are of small diameter and minimal in quantity. This visual observation corresponds to a measured differential pressure that contains very little noise or fluctuation in the differential pressure value that was observed during good fluidization or non-sticky conditions. Under non-sticky operating conditions, a typical pressure drop of about 78±2 mbar was observed. All the catalysts were tested under the same conditions so that a direct comparison of the results was ensured.

### Example 1

Catalysts were prepared by impregnation of an alumina support from Sassol (Catalox® SCCa-25/200) with aqueous metal chloride solution, which is prepared by dissolving of copper chloride (CuCl₂·2H₂O), magnesium chloride (MgCl₂·6H₂O), potassium chloride (KCl₂·2H₂O), and zirconium oxychloride (ZrOCl₂·8H₂O) in water at 60° C. under vigorous stirring. The used support is characterized with a surface area of 180 to 200 m²/g, a pore volume of 0.4 to 0.5 cm³/g, and a particle size distribution of about 20 μm to about 100 μm (ca. 4.0% of the particles were smaller than 22 μm, ca. 10% of the particles were smaller than 31 μm, ca. 29% of the particles were smaller than 44 μm, ca. 85% of the particles were smaller than 88 μm, and ca. 98% of the particles were smaller than 125 μm). In addition, the support material contains about 0.1 to 0.2% of Ti, expressed in metal form, as impurity from the production process. The metal composition of the prepared catalyst is 4.5 wt. % Cu, 1.9 wt. Mg and 0.25 wt. % K and 1.0 wt. % Zr.

### Example 2

Metal chlorides, such as Cu, Mg and Zr, are impregnated on Catalox® SCCa-25/200 using the same raw materials like Example 1. The metal composition is 4.5 wt. % Cu, 2.0 wt. % Mg and 1.0 wt. % Zr.

### Example 3

Metal chlorides are impregnated on Catalox® SCCa-25/200. The metal composition is 4.5 wt. % Cu, 1.6 wt. % Mg and 1.0 wt. % Zr.

### Example 4

Metal chlorides are impregnated on Catalox® SCCa-25/200. The metal composition is 4.5 wt. % Cu, 1.0 wt. % Mg and 1.0 wt. % Zr.

### Example 5

Metal chlorides are impregnated on Catalox® SCCa-25/200. The metal composition is 4.5 wt. % Cu, 1.3 wt. % Mg and 1.0 wt. % Zr.

### Comparative Example 1

Metal chlorides are impregnated on Catalox® SCCa-25/200. The metal composition was 4.15 wt. % Cu and 2.12 wt. % Mg.

### Comparative Example 2

Metal chlorides are impregnated on Catalox® SCCa-25/200. The metal composition was 4.3 wt. % Cu, 1.3 wt. % Mg, 1.0 Mn and 1.0 wt. % K.

### Comparative Example 3

The catalysts were prepared as Example 1, however, Zr content was excluded in metal chloride solution. The metal composition is 4.5 wt. % Cu, 1.9 wt. % Mg and 0.25 wt. % K.

### Comparative Example 4

The catalysts were prepared as Example 5, however, Zr content was excluded in metal chloride solution. The metal composition is 4.5 wt. % Cu and 1.3 wt. % Mg.

TABLE 1
Elemental composition of Catalysts

| | Cu | Mg | Mn | K | Zr |
|---|---|---|---|---|---|
| Ex 1 | 4.5 | 1.9 | — | 0.25 | 1.0 |
| Ex 2 | 4.5 | 1.9 | — | — | 1.0 |
| Ex 3 | 4.5 | 1.6 | — | — | 1.0 |
| Ex 4 | 4.5 | 1.0 | — | — | 1.0 |
| Ex 5 | 4.5 | 1.3 | — | — | 1.0 |
| Comp Ex 1 | 4.15 | 2.12 | — | — | — |
| Comp Ex 2 | 4.3 | 1.3 | 1.0 | 1.1 | |
| Comp Ex 3 | 4.5 | 1.9 | — | 0.25 | — |
| Comp Ex 4 | 4.5 | 1.3 | — | — | — |

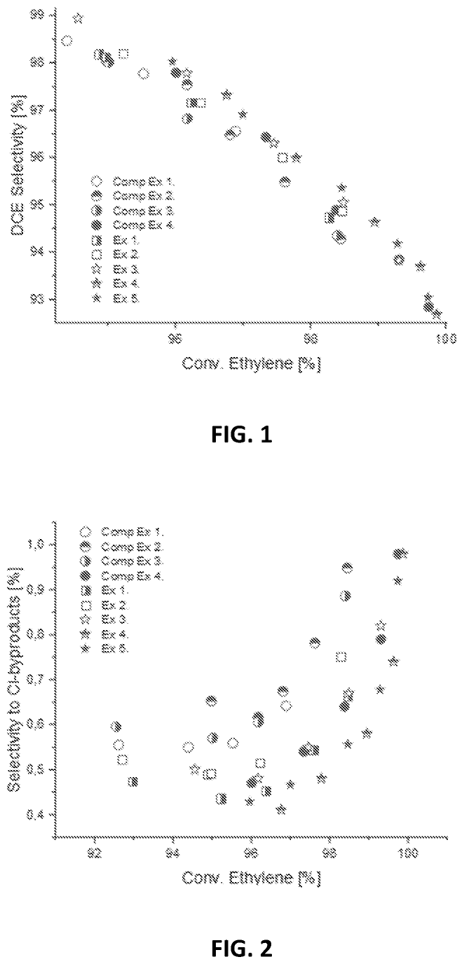

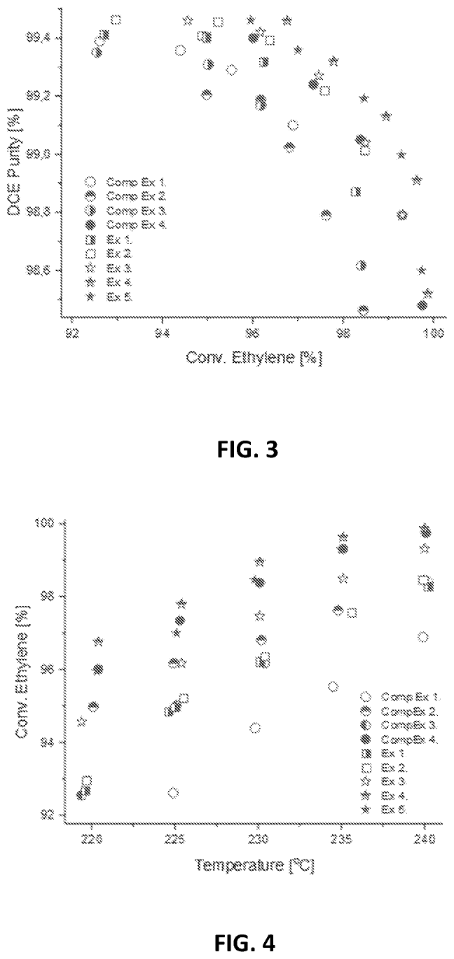

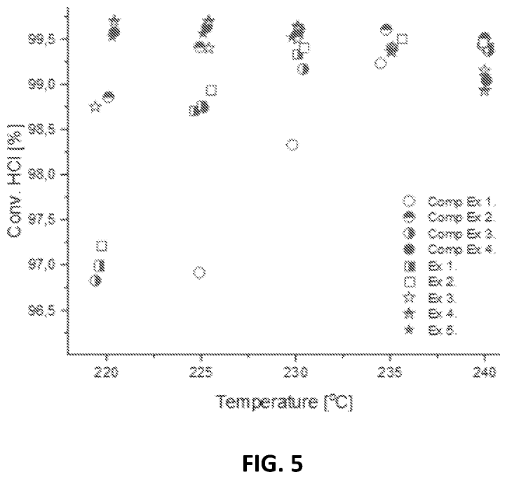

Catalytic performances of all inventive and comparative Examples, measured between 220-240° C., are plotted in FIGS. 1-5. In FIG. 1, increased DCE selectivity (vs. ethylene conversion) by addition of 1.0 wt. % Zr to catalyst composition is revealed by comparing Ex 1 and Comp Ex 3 while activity of Ex 1 remains as comparable as Comp Ex 3 (FIGS. 4 and 5). As shown in FIG. 2, the improved DCE product purity for Ex 1 is mainly attributed to reduced selectivity toward, but not limited, chlorinated by-products. As a result, DCE product purity of Ex 1 is ca. 0.2 percent point higher than that of Comp Ex 3 throughout overall ethylene conversion (Error! Reference source not found).

DCE product purity (vs. ethylene conversion) can be also improved by modification of the amount of Mg content in catalyst composition, for example, reducing Mg content from 1.9 to 1.0 wt. % (Ex 2. to Ex 5. in Error! Reference source not found). However, in this case, improved DCE purity (vs. ethylene conversion) is largely due to increased activity of catalyst at lower operation temperature (<230° C.), at which selectivities to chlorinated by-products are reduced.

The influence of Zr compound on selectivity to chlorinated by-products, thereby DCE purity, is once again recognized when catalytic performance of Ex 5 is compared to that of Comp Ex 4, which has the same catalyst formulation of Ex 5 except Zr content. Both samples show comparable catalytic activity, however, DCE purity of Ex 5 is ca. 0.1-0.2 percent point higher than that of Comp Ex 4 as shown in previous comparison between Ex 1 and Comp Ex 3. As a result, Zr content is considered a promoter that can improve DCE product purity without adjusting catalyst activity in oxychlorination of ethylene to 1,2-dichloroethane.

Additionally, inventive Examples, introduced above, illustrate superior DCE purity in comparison with Comp. Ex 1 as well as Comp. Ex 2.

Stickiness test, following activity test of corresponding catalyst by lowering reaction temperature by 2 or 3° C./step, reveals that inventive examples show sufficient fluidizability down to 195° C. under the reaction condition. This indicates that some of inventive Examples can be applicable to a commercial plant operating at <230° C.

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail in order to avoid unnecessarily obscuring the present invention. Thus, the specific details set forth are exemplary. Particular embodiments may vary from these exemplary details and still be contemplated to be within the scope of the present invention.

Although the operations of the methods herein are described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

1. A catalyst composition comprising:
a support; and
catalytic metal compounds on the support, the catalytic metal compounds comprising:
a copper compound; and
a zirconium compound,
wherein the support has a BET surface of about 50 m²/g to about 250 m²/g.

2. The catalyst composition of claim 1, wherein the support comprises alumina.

3. The catalyst composition of claim 1, wherein the support comprises gamma alumina.

4. The catalyst composition of claim 1, wherein the catalytic metal compounds are impregnated in the support.

5. The catalyst composition of claim 1, wherein the catalytic metal compounds are adsorbed onto the support.

6. The catalyst composition of claim 1, wherein the copper compound comprises a copper salt.

7. The catalyst composition of claim 6, wherein the copper salt is selected from a group consisting of a copper chloride, a copper nitrate, a copper bromate, a copper carbonate, a copper sulfate and combinations thereof.

8. The catalyst composition of claim 1, wherein the catalytic metal compounds further comprise at least one alkaline earth metal compound comprising magnesium.

9. The catalyst composition of claim 8, wherein the at least one alkaline earth metal compound comprises a magnesium salt.

10. The catalyst composition of claim 9, wherein the magnesium salt is selected from a group consisting of magnesium chloride, magnesium nitrate, magnesium bromate, magnesium carbonate, magnesium sulfate and combinations thereof.

11. The catalyst composition of claim 1, wherein the zirconium compound comprises a zirconium salt.

12. The catalyst composition of claim 11, wherein the zirconium salt is selected from a group consisting of zirconium oxychloride, zirconium chloride, zirconium carbonate, zirconium nitrate, zirconium hydroxide and combinations thereof.

13. The catalyst composition of claim 1, comprising about 1.0 wt. % to about 10.0 wt. % of copper metal based on the total weight of the catalyst composition.

14. The catalyst composition of claim 1, wherein the catalytic metal compounds further comprise about 0.5 wt. % to about 5.0 wt. % of magnesium metal based on the total weight of the catalyst composition.

15. The catalyst composition of claim 1, comprising about 0.1 wt. % to about 3.0 wt. % of zirconium metal based on the total weight of the catalyst composition.

16. The catalyst composition of claim 1, wherein the catalyst composition has an ethylene dichloride selectivity of about 95 mol % to about 99.9 mol % when contacted with hydrochloric acid, ethylene and oxygen at a temperature of about 200° C. to about 250° C.

17. The catalyst composition of claim 1, wherein the catalyst composition provides a dichloroethane crude purity of about 99.2 wt % to about 99.9 wt % when used for the oxychlorination of ethylene.

18-21. (canceled)

22. A catalyst composition comprising:
a support; and
catalytic metal compounds on the support, the catalytic metal compounds comprising:
a copper compound; and
a zirconium compound,
wherein the support comprises a particle size distribution such that (a) about 90 percent to about 100 percent by volume of the particles are less than about 150 μm, (b) about 50 percent to about 60 percent by volume of the particles are less than about 75 μm; and (c) about 20 percent to about 35 percent by volume of the particles are less than about 45 μm, as measured at about 20° C. to about 25° C. and about 1 atm by a Malvern Instruments laser diffraction analyzer wherein a 5 g sample of the support is dispersed in 100 mL of deionized water with a magnetic stirrer for 1 min to form a suspension and 4 mL of the suspension is diluted with 130 mL of deionized water and analyzed at 2500 rotations/min with the analyzer.

23-41. (canceled)

42. A method of forming a catalyst composition, comprising:
dissolving a copper compound and at least one compound comprising zirconium in a solvent with agitation and at a temperature of about 20° C. to about 80° C. to form a solution;
dosing the solution onto a support comprising a BET surface of about 50 m²/g to about 250 m²/g, wherein the support is rotated at about 50 rotations per minute (rpm) to about 150 rpm; and
drying the coated support to remove free water for about 1 hour to about 20 hours at a temperature of about 75° C. to about 180° C. to form the catalyst composition.

43-62. (canceled)

63. A method of using a catalyst composition according to claim 1, the method comprising:
converting ethylene to dichloroethane using an oxychlorination process in the presence of the catalyst composition.

64. (canceled)

65. (canceled)
